(19) United States
(12) Patent Application Publication
(10) Pub. No.: US 2017/0049416 A1
(43) Pub. Date: Feb. 23, 2017

(54) ELASTOGRAPHY VISUALIZATION

(71) Applicant: B-K MEDICAL APS, Herlev (DK)

(72) Inventors: Reza Z. AZAR, Vancouver (CA); Kris DICKIE, Vancouver (CA); Michelle ALEXANDER, Lithia Springs, GA (US)

(73) Assignee: B-K Medical Aps, Herlev (DK)

(21) Appl. No.: 15/308,197

(22) PCT Filed: May 2, 2014
(86) PCT No.: PCT/IB2014/061165

§ 371 Date: Nov. 1, 2016

(51) Int. Cl. A61B 8/00; A61B 8/08; A61B 90/30

(52) U.S. Cl. CPC A61B 8/4455; A61B 8/4483; A61B 90/30; A61B 8/485; A61B 8/5207; A61B 2090/306

(57) ABSTRACT

A method includes receiving a B-mode image, a strain image, and a corresponding correlation image. The method further includes modifying pixel values of the strain image based on a reliability of the strain image, thereby generating a modified strain image. The method further includes displaying the B-mode image. The method further includes superimposing the modified strain image over the B-mode image. A system (100) includes a memory (304) that stores elastography visualization algorithms (306, 308, 310). The system further includes a processor (302) that executes at least one of the elastography visualization algorithms, based on a visualization mode of interest, causing the processor to render at least one of a pixel of a strain image transparent or not at all, wherein the strain image is displayed overlaid over a B-mode image.

### TECHNICAL FIELD

The following generally relates to elastography visualization and more particularly to ultrasound elastography image visualization, and is described with particular application to an ultrasound imaging system.

### BACKGROUND

An ultrasound imaging system has included at least an ultrasound probe and a console. The ultrasound probe houses a transducer array of transducing elements, and the console includes a display monitor and a user interface. The transducing elements transmit an ultrasound signal into a field of view and receive echoes produced in response to the signal interacting with structure therein. In B-mode, the echoes are processed, producing a sequence of focused, coherent echo samples along focused scanlines of a scanplane. The scanlines are scan converted into a format of a display monitor and visually presented as image via the display monitor.

With ultrasound elastography imaging, or real-time strain imaging, ultrasound images are acquired from the tissue while the tissue undergoes compression or deformation. The compression or deformation can be applied manually by the user (i.e. by slightly pressing the transducer against the tissue), induced by internal tissue motions (due to breathing or heart beat), or induced through focused beams of ultrasound energy to produce movement. During the compression cycle, ultrasound signals are acquired and processed to generate the corresponding strain images. The strain images have been displayed, for example, as a measure of tissue elasticity along-side the B-mode images.

Strain images, generally, are a function of the applied compression. As a consequence, small compressions may not generate enough contrast and will lower the detectability of the tissue abnormalities, while large compressions may result in unreliable measurements and invalid images. In addition to the compression, strain images are also a function of underlying tissue structure, as well as their ultrasonic signal to noise ratio. As such, some of the displayed pixels may not represent valid or useful information. Unfortunately, this may result in a false interpretation of the image.

### SUMMARY

Aspects of the application address the above matters, and others.

In one aspect, a method includes receiving a B-mode image, a strain image, and a corresponding correlation image. The method further includes modifying pixel values of the strain image based on a reliability of the strain image, thereby generating a modified strain image. The method further includes displaying the B-mode image. The method further includes superimposing the modified strain image over the B-mode image.

In another aspect, a system includes a memory that stores elastography visualization algorithms. The system further includes a processor that executes at least one of the elastography visualization algorithms, based on a visualization mode of interest, causing the processor to render at least one of a pixel of a strain image transparent or not at all, wherein the strain image is displayed overlaid over a B-mode image.

In another aspect, an ultrasound imaging system includes a transducer array of transducer elements. The ultrasound imaging system further includes transmit circuitry that generates a pulse that excites at least a sub-set of the transducer elements to transmit an ultrasound signal in a field of view. The ultrasound imaging system further includes receive circuitry that receives echoes, which are generated in response to the ultrasound signal interacting with structure in the field of view. The ultrasound imaging system further includes an echo processor that processes the echoes, generating a B-mode image. The ultrasound imaging system further includes an elastography processor that processes the echoes, generating a strain image and a corresponding correlation image.

The ultrasound imaging system further includes a rendering engine that renders the strain image over the B-mode image, wherein the rendering engine renders the strain image based on at least one of a soft blend algorithm, a hard blend algorithm, or a B-mode priority algorithm. The soft blend algorithm causes the rendering engine to render a pixel of the strain image using a transparency level, the hard blend algorithm causes the rendering engine to render the pixel of the strain image completely transparent, and the B-mode priority algorithm causes the rendering engine to ignore the pixel of the strain image.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example ultrasound imaging system with an elastogram processor and a rendering engine;

FIG. 2 schematically illustrates an example of the elastogram processor;

FIG. 3 schematically illustrates an example of the rendering engine;

FIG. 4 shows an example B-mode image;

FIG. 5 shows an example strain image;

FIG. 6 shows an example of the strain image of FIG. 5 superimposed over the B-mode image of FIG. 4;

FIG. 7 shows an example of a soft blend strain image;

FIG. 8 shows an example of the soft blend strain image of FIG. 7 superimposed over the B-mode image of FIG. 4;

FIG. 9 shows an example of a hard blend strain image;

FIG. 10 shows an example of the hard blend strain image of FIG. 9 superimposed over the B-mode image of FIG. 4;

FIG. 11 shows an example in which strain image pixels corresponding to B-mode image pixels generated with not enough signal are not displayed or are displayed completely transparent over the B-mode image of FIG. 4;

FIG. 12 illustrates an example method in accordance with the embodiments disclosed herein;

FIG. 13 illustrates example ultrasound imaging system in accordance with the embodiments disclosed herein; and

FIG. 14 illustrates another example ultrasound imaging system in accordance with the embodiments disclosed herein.

### DETAILED DESCRIPTION

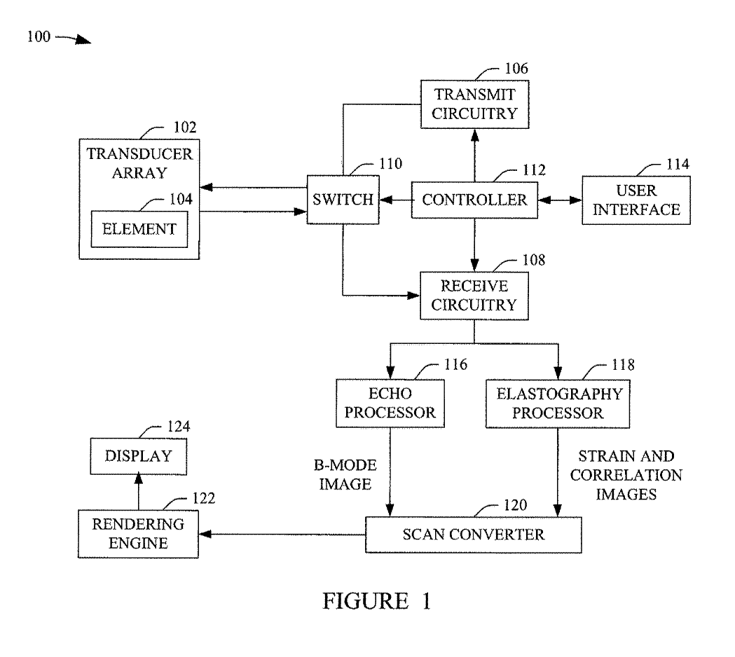

FIG. 1 illustrates an example imaging system 100, such as an ultrasound imaging system.

The ultrasound imaging system 100 includes a transducer array 102. The transducer array 102 can include a one dimensional (1D) or two dimensional (2D) array of transducer elements 104. The transducer elements 104 are configured to transmit ultrasound signals and receive echo signals. Suitable arrays 102 include linear, curved, and/or otherwise shaped. The transducer array 102 can be fully populated or sparse.

The ultrasound imaging system 100 includes transmit circuitry 106. The transmit circuitry 106 generates a set of radio frequency (RF) pulses that are conveyed to the transducer array 102. The set of pulses actuates a corresponding set of the transducer elements 104, causing the elements to transmit ultrasound signals into an examination or scan field of view. For elastography imaging, compression/deformation is applied manually by the user, through focused beams, induced internally by heartbeat or breathing, etc. during transmit of ultrasound signals.

The ultrasound imaging system 100 includes receive circuitry 108. The receive circuitry 108 receives echoes (RF signals) generated in response to the transmitted ultrasound signals from the transducer array 102. The echoes, generally, are a result of the interaction between the emitted ultrasound signals and the structure (e.g., flowing blood cells, organ cells, etc.) in the scan field of view. For elastography imaging, frames are acquired continuously at a rate of up to 30 frames per second or higher.

The ultrasound imaging system 100 includes a switch 110. The switch 110 switches between the transmit circuitry 106 and the receive circuitry 108, depending on whether the transducer array 102 is operated in transmit or receive mode. In transmit mode, the switch 110 electrically connects the transmit circuitry 106 to the elements 104. In receive mode, the switch 110 electrically connects the receive circuitry 108 to the elements 104.

The ultrasound imaging system 100 includes a controller 112. The controller 112 controls one or more of the transmit circuitry 106, the receive circuitry 108 or the switch 110. Such control can be based on available modes of operation. Examples of such modes of operation include one or more of B-mode, elastography mode, A-mode, velocity flow mode, Doppler mode, etc.

The ultrasound imaging system 100 includes a user interface (UI) 114. The UI 114 may include one or more input devices (e.g., a button, a knob, a slider, a touch pad, etc.) and/or one or more output devices (e.g., a display screen, lights, a speaker, etc.). The UI 114 can be used to select an imaging mode, activate scanning, etc.

The ultrasound imaging system 100 further includes an echo processor 116 that processes received echoes. Such processing may include applying time delays, weighting on the channels, summing, and/or otherwise beamforming received echoes. In B-mode, the echo processor 116 produces a sequence of focused, coherent echo samples along focused scanlines of a scanplane. Other processing may lower speckle, improve specular reflector delineation, and/or includes FIR filtering, IIR filtering, etc.

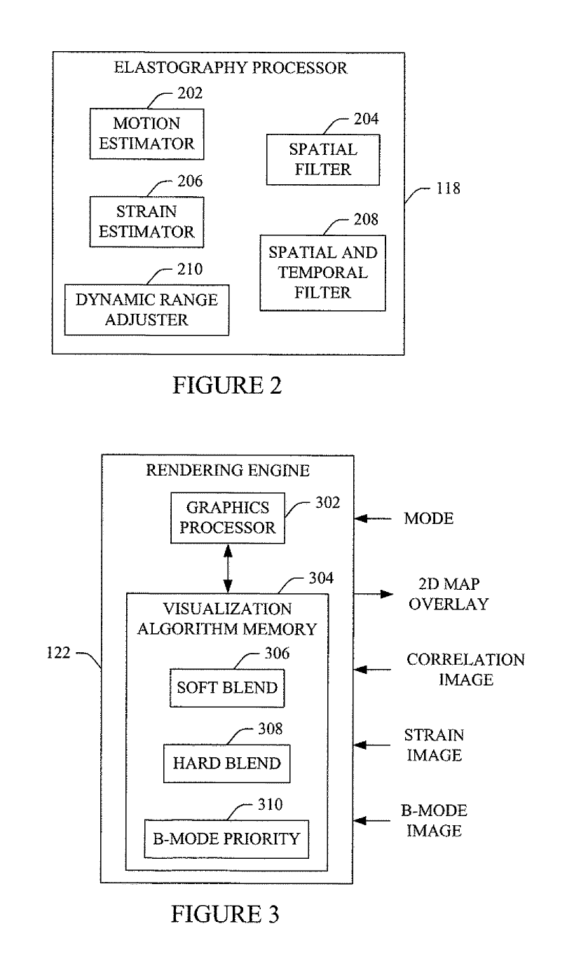

The elastogram processor 118 processes the received signals and generates a strain image between at least two sequential frames, and a corresponding correlation image(s). In a variation, the elastogram processor 118 processes the B-mode images from the echo processor 116 and generates the strain image and the corresponding correlation image. Briefly turning to FIG. 2, a non-limiting example of the elastogram processor 118 is schematically illustrated.

The elastogram processor 118 includes a motion estimator 202. The motion estimator 202 estimates motion between sequences of received signals. In one non-limiting instance, this includes dividing the signals into windows of small overlapping windows and applying a motion tracking algorithm to each window. Elastograms with different resolutions can be generated by adjusting the size and overlap between these windows. Known and/or other motion algorithms can be employed. The motion estimator 202 outputs a displacement image.

The motion estimator 202 also outputs a corresponding correlation image. The displacement image represents the displacement between successive windows, and correlation image indicates a degree of match or similarity between the corresponding windows. In a non-limiting example, the normalized correlation image includes values between minus one (−1) and one (1), which indicates reliable sub-portions of the displacement image (e.g. sub-portions corresponding to higher correlation values) and less reliable and/or unreliable sub-portions of the displacement image (e.g. sub-portions corresponding to lower correlation values). Generally, −1 means the windows are inverted version of each other, 0 means no similarity, and 1 means they are perfect match. It is understood that alternative measures of similarity such as non-normalized correlation image, sum of absolute differences, sum of absolute differences etc. as well as techniques that use phase shift estimation and complex correlation are contemplated herein.

The elastogram processor 118 further includes a spatial filter 204. The spatial filter 204 applies a 2D spatial filtering to the displacement image and the correlation image. Suitable filters include a 2D medial filterer, a 2D mean filterer, and/or other filter. The 2D medial filterer, for example, removes outliers. The 2D mean filterer, for example, improves the signal to noise ratio (SNR). In a variation, the spatial filter 204 is omitted from the elastogram processor 118.

The elastogram processor 118 further includes a strain estimator 206. The strain estimator 206 processes the displacement images and generates strain images. The strain estimator 206 can employ known and/or other strain estimation algorithms. An example of a known strain estimation algorithm includes a least-squares strain estimator.

The elastogram processor 118 further includes spatial and temporal filter 208. The spatial and temporal filter 208 applies spatial filtering and temporal persistency to both the strain images and the correlation images. This, for example, improves the SNR of both the strain images and the correlation images. The spatial and temporal filter 208 can employ known and/or other strain estimation algorithms In a variation, the spatial and temporal filter 208 is omitted from the elastogram processor 118.

The elastogram processor 118 further includes a dynamic range adjuster 212. The dynamic range adjuster 210 maps the strain values a predetermined scale such as the full scale other scale. In one instance, the mapping maintains the maximum dynamic range of the strain values. Examples of suitable algorithms include contrast stretching, histogram equalization, and/or other algorithms

Returning to FIG. 1, the ultrasound imaging system 100 further includes a scan converter 120. The scan converter 120 scan converts the output of the echo processor 116 and generates data for display, for example, by converting the data to the coordinate system of a display. The scan converter 120 also scan converts both the strain image and correlation images based on the geometry of the B-mode image. In a variation, a different scan converter is used for the B-mode image and the strain and correlation images.

The ultrasound imaging system 100 includes a rendering engine 122 and a display 124. The rendering engine 122 visually presents the B-mode image overlaid with the strain image. In one instance, the rendering engine 122 overlays only the strain image (e.g., a color-coded or gray scale) over the B-mode image. In this instance, the strain image is a 1D map that maps the elasticity or strain values directly to the B-mode image.

In another instance, the rendering engine 122 creates a 2D overlay image based on the strain image and the correlation image and/or the B-mode image. As described in greater detail below, the overly image takes into account the reliability of each pixel of the strain image and gradually or abruptly visually suppresses (e.g., ignores, renders transparent, etc.) strain image pixels as a function of the reliability. This may include taking into account the signal used to create a B-mode image pixel, and visually suppressing the strain image pixel based on the reliability of the B-mode image pixel.

As such, the observer of the combined image B-mode/strain image will be apprised of whether a strain image pixel corresponds to a valid or suspect measurement, which may facilitate mitigating a false interpretation of the B-mode image. As a consequence, the displayed data can be used, for example, during the training phase when the clinicians are trying to improve their scanning techniques, during live scan to provide feedback to an end user such that the end user knows when a good sequence of elastograms have been acquired, during exam review when selecting individual frames where good images are generated, etc.

This also allows, in one non-limiting instance, the clinician to better evaluate the generated images in real-time (as the data is acquired and images are generated) and/or off-line during the exam review, relative to just displaying the strain image alone over a B-mode image. It also allows the user to improve their scanning techniques and acquire better strain images. Generally, the processing of the rendering engine 122 improves visualization of strain images. It also provides for calculation of quality feedback to help clinicians acquire repeatable and more reliable strain images.

It is to be appreciated that one or more of the echo processor 116, the elastogram processor 118, and the rendering engine 122 can be implemented via a processor (e.g., a microprocessor, central processing unit, etc.) executing one or more computer readable instructions encoded or embedded on computer readable storage medium, such as physical memory.

Turning to FIG. 3, an example of the rendering engine 122 is schematically illustrated.

The rendering engine 122 includes a graphics processor 302 and a visualization algorithm memory 304. The graphics processor 302 receives, as input, a signal from the controller 112 (FIG. 1). The signal indicates the visualization mode. The visualization mode can be determined based on a predetermined default mode, a user input through the UI 114 (FIG. 1), and/or otherwise. The graphics processor 302 also receives one or more of the B-mode image, the strain image and/or the correlation image.

The graphics processor 302, based on the visualization mode, retrieves and/or invokes a suitable algorithm from the visualization algorithm memory 304. In the illustrated embodiment, the visualization algorithm memory 304 stores at least one of a soft blend 306 algorithm, a hard blend 308 algorithm or a B-mode priority 310 algorithm. Alternatively, the graphics processor 302 displays the B-mode image with the strain image, unmodified, overlaid there over. For this, the strain image can be considered a 1D map in that it provides only strain information, which is mapped directly to the B-mode image.

With the soft blend 306 algorithm, pixels of the strain image corresponding to lower correlation values in the correlation image are rendered more transparent, and pixels of the strain image corresponding to higher correlation values in the correlation image are rendered opaque or less transparent. In this mode, the graphics processor 302 identifies a correlation value of a pixel from the correlation image that corresponds to the strain image pixel being processed. The graphics processor 302 then identifies a transparency level for the correlation value. This can be through a look up table (LUT), a mathematical function (e.g., a polynomial), and/or otherwise. The graphics processor 302 renders the pixel with the transparency level.

The transition in transparency from a correlation value of zero (or some other value) to a correlation value of one (or some other range) can be linear, non-linear, or have both linear and non-linear regions. The transition in transparency can also be continuous, discrete or have both continuous and discrete regions. With this mode, a user sees less strain image and more the B-mode image (in the background) as the correlation value approaches zero and more strain image and less the B-mode image (in the background) as the correlation value approaches one. The soft blend strain image can be considered a 2D image in that it provides strain information and strain reliably information.

With the hard blend 308 algorithm, strain image pixels with a corresponding correlation value less than a predetermined threshold are rendered transparent, and all other pixels are rendered opaque (or less transparent). In this mode, the graphics processor 302 identifies the correlation value of a pixel from the correlation image that corresponds to the strain image pixel being processed. The graphics processor 302 the compares the correlation value with a predetermined threshold.

The graphics processor 302 then makes a binary decision as to whether to show the pixel or not (or display it transparent). The graphics processor 302 renders the pixel accordingly. This mode causes a sub-part of a strain image corresponding to low correlation to be completely removed or hidden. The hard blend strain image, like the soft blend strain image, can be considered a 2D image in that it provides strain information and strain reliably information.

With the B-mode priority 310 algorithm, the graphics processor 302 compares a B-mode image pixel value with a predetermined threshold. If the pixel value is less than the predetermined threshold, the strain image pixel corresponding to the B-mode image pixel is not displayed or is displayed transparent. Otherwise, the strain image pixel is displayed or is displayed, for example, based on the soft blend 306 algorithm, the hard blend 308 algorithm, and/or otherwise.

With this algorithm, dark regions in the B-mode image are displayed as B-mode only, with no strain overlay. This ensures that elastography data are not displayed when there is not enough of a predetermined amount of ultrasound signal. The reason for this is that the strain values in such regions may not be valid and/or reliable due to lack of signal. The B-mode priority strain image, like the soft blend strain image and the hard blend strain image, can be considered a 2D image in that it provides strain information and strain reliably information.

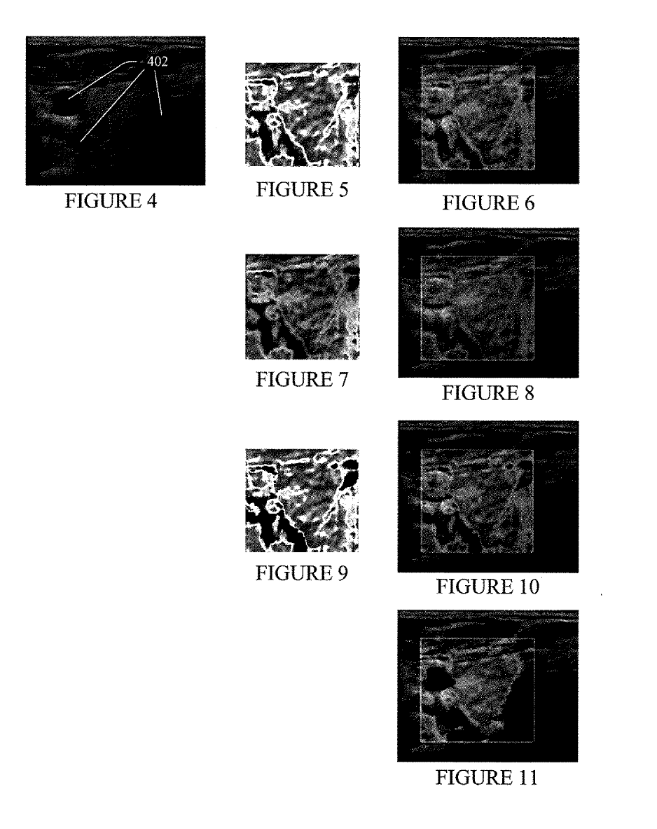

FIG. 4 show an example of a B-mode image. FIG. 5 shows an example of a strain image. FIG. 6 shows an example of the B-mode image of FIG. 4 with the strain image of FIG. 5 superimposed there over. In FIG. 6, the strain image is superimposed on the B-mode image, and the correlation image is ignored. With FIG. 6, invalid or poor strain measurements are displayed and shown to the end user. This may result in false interpretation of the image. In this example, the strain image is shown in gray scale. However, it is to be understood that the strain image can be shown using a color map.

FIG. 7 shows an example soft blend strain image. As described herein, the rendering engine 122 generates a soft blend strain image by applying a transparency map to the strain image. For this, the rendering engine 126, for a pixel in the strain image, identifies a corresponding pixel in the correlation image. A correlation value is identified for the identified pixel. A transparency level is then identified for the identified correlation value. The transparency level is then applied to the pixel in the strain image. This is repeated for all the pixels in the strain image. FIG. 8 shows an example of the B-mode image of FIG. 4 with the soft blend strain image of FIG. 7 superimposed there over.

FIG. 9 shows an example hard blend strain image. As described herein, the rendering engine 122 generates a hard blend image by thresholding the strain image. For this, the rendering engine 126, for a pixel in the strain image, identifies a corresponding pixel in the correlation image. A correlation value is identified for the identified pixel. The correlation value is compared against a predetermined threshold. If the correlation value is below the predetermined threshold, the strain image pixel value is set to completely transparent. Otherwise, the strain image pixel value is set with a transparency level from semi-transparent to opaque. This is repeated for all the pixels in the strain image. FIG. 10 shows an example of the B-mode image of FIG. 4 with the hard blend strain image of FIG. 9 superimposed there over.

FIG. 11 shows an example B-mode priority image. As described herein, the rendering engine 122 identifies, for a pixel in the B-mode image, a pixel value. The rendering engine 122 thresholds the B-mode image pixel value. For this, the rendering engine 126 compares the B-mode pixel value with a predetermined threshold. If the B-mode pixel value is below the predetermined threshold, the strain image pixel value is set to completely transparent or ignored. Otherwise, the strain image pixel value is set with a transparency level from semi-transparent to opaque.

This is repeated for all the pixels in the B-mode image and all the corresponding pixels in the strain image. FIG. 11 shows an example of the B-mode image of FIG. 4 with the only pixels of strain image of FIG. 4 with values satisfying the threshold superimposed there over. The pixels of strain image of FIG. 4 with values that do not satisfy the threshold are not superimposed there over. In the illustrated example, darker region 402 (FIG. 4) represent regions of low signal, and do not satisfy the predetermined threshold, and the strain image pixels corresponding to thereto are not shown.

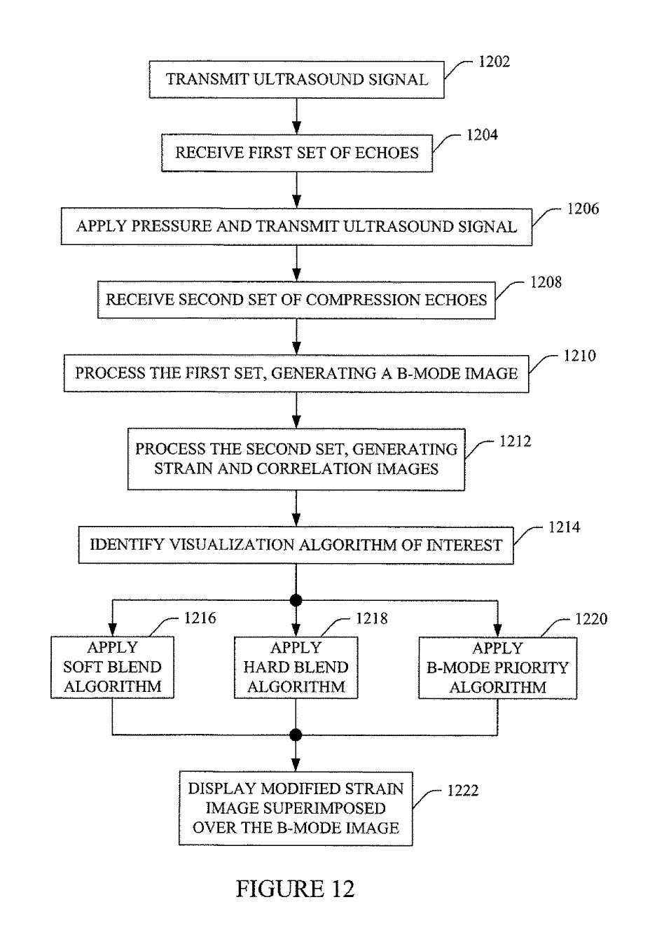

FIG. 12 illustrates a method.

It is to be appreciated that the order of the following acts is provided for explanatory purposes and is not limiting. As such, one or more of the following acts may occur in a different order. Furthermore, one or more of the following acts may be omitted and/or one or more additional acts may be added.

At 1202, an array of ultrasound transducer elements is placed again a surface of a subject or object and activated to transmit an ultrasound signal into a field of view.

At 1204, a first set of echoes is received.

At 1206, pressure is applied to the subject or object. As discussed herein, this can be achieved by manually applying a pressure to the surface via the array, applying focused beams of ultrasound energy to the subject or object, etc.

At 1208, a second set of echoes (compression echoes) is received. As described herein, the second set of echoes may include two or more sets of echoes.

At 1210, the first set of echoes is processed, generating a B-mode image.

At 1212, the second set of echoes is processed, generating a strain image and a correlation image. As described herein, the strain image and corresponding correlation image(s) may be generated from two or more sets of the second set of echoes.

For real-time imaging, the echoes are acquired continuously, and for each new acquisition, one B-mode image and one elastography image (e.g., by buffering the previous data) are generated.

At 1214, an elastogram visualization algorithm is retrieved based on an elastogram mode of operation of interest. As discussed herein, the mode can be a default, user specified, changed, etc.

At 1216, in response to the mode of operation of interest being a soft blend mode, the soft blend 306 algorithm is applied. For this, for a pixel in the strain image, a corresponding pixel in the correlation image is identified. A correlation value is identified for the identified pixel. A transparency level is then identified for the identified correlation value. The transparency level is then applied to the pixel in the strain image. This is repeated for other pixels in the strain image.

At 1218, in response to the mode of operation of interest being a hard blend mode, the hard blend 308 algorithm is applied. For this, for a pixel in the strain image, a corresponding pixel in the correlation image is identified. A correlation value is identified for the identified pixel. The identified correlation pixel value is compared against a predetermined threshold. If the correlation pixel value is less than the threshold, the corresponding strain image pixel is set to completely transparent or ignored. Otherwise, the corresponding strain image pixel is set to semi-transparent or opaque. This is repeated for other pixels in the strain image.

At 1220, in response to the mode of operation of interest being a B-mode priority, the B-mode priority 310 algorithm is applied. For this, for a pixel in the B-mode image, a pixel is identified. The identified B-mode image pixel value is compared against a predetermined threshold. If the B-mode image pixel value is less than the threshold, the corresponding strain image pixel is set to completely transparent or ignored. Otherwise, the corresponding strain image pixel is set to semi-transparent or opaque. This is repeated for other pixels in the B-mode image.

At 1222, the modified strain image is displayed, superimposed over the B-mode image.

At least a portion of the method discussed herein may be implemented by way of computer readable instructions, encoded or embedded on computer readable storage medium (which excludes transitory medium), which, when executed by a computer processor(s), causes the processor(s) to carry out the described acts. Additionally or alternatively, at least one of the computer readable instructions is carried by a signal, carrier wave or other transitory medium.

Generally, the embodiments described herein provide only valid and informative elastograms to the end user, for example, by making invalid regions of the strain image slightly or completely transparent, and/or blocking the elastography from displaying regions of the strain image that corresponds to regions of the B-mode image where not enough signal was acquired, for example, dark regions in the B-mode image.

The embodiments can be used, for example, during the training phase when the clinicians are trying to improve their scanning techniques, during live scan to provide feedback to an end user such that the end user knows when a good sequence of elastograms have been acquired, during exam review when selecting individual frames where good images are generated, etc.

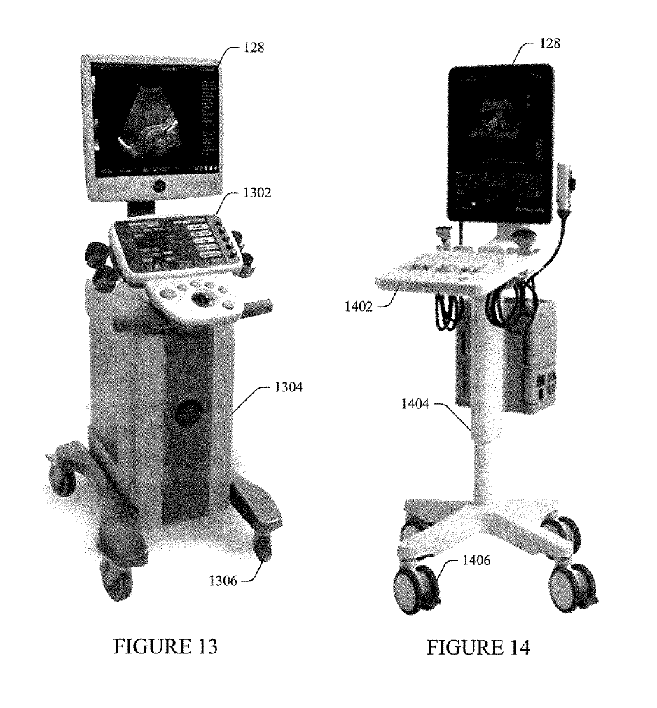

FIGS. 13 and 14 illustrate non-limiting examples of the ultrasound imaging system 100. In the non-limiting examples, at least the echo processor 116, the elastography processor 118, the scan converter 120, and the rendering engine 122 are part of console 1302 and 1402, and the display 124 and the console 1302 and 1402 are integrated in and part of respective mobile carts 1304 and 1404, which include movers 1306 and 1406 such as wheels, casters, etc.

In another configuration, the ultrasound imaging system 100 does not include movers and/or is not integrated into a cart, but instead rests on a table, desk, etc. In another configuration, the ultrasound imaging system 100 is part of a hand-held ultrasound scanner. An example of a hand-held scanner is described in U.S. Pat. No. 7,699,776, entitled “Intuitive Ultrasonic Imaging System and Related Method Thereof,” and filed on Mar. 6, 2003, which is incorporated herein in its entirety by reference.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A method, comprising:
receiving a B-mode image, a strain image, and a corresponding correlation image;
modifying pixel values of the strain image based on a reliability of the strain image, thereby generating a modified strain image;
displaying the B-mode image; and
superimposing the modified strain image over the B-mode image.

2. The method of claim 1, further comprising:
modifying the pixel values of the strain image based on at least one of pixel values of the correlation image or pixel values of the B-mode image, thereby generating the modified strain image.

3. The method of claim 1, further comprising:
identifying a pixel of the B-mode image;
identifying a pixel of the correlation image that corresponds to the pixel of the strain image;
identifying a pixel value for the identified pixel of the correlation image, wherein the pixel value is a correlation value in a predetermined range;
retrieving a predetermined transparency value for the identified correlation value;
applying the transparency value to the pixel of the strain image, creating a modified strain image pixel; and
superimposing the modified strain image pixel over the corresponding B-mode image pixel.

4. The method of claim 3, further comprising:
scaling the transparency value as a function of the correlation value of the pixel of the correlation image.

5. The method of claim 4, wherein the scaling is continuous.

6. The method of claim 4, wherein the scaling is discrete.

7. The method of claim 4, wherein the scaling is linear.

8. The method of claim 4, wherein the scaling is non-linear.

9. The method of claim 3, further comprising:
retrieving the transparency value from a look up table that maps transparency values to correlation values.

10. The method of claim 1, further comprising:
identifying a pixel of the B-mode image;
identifying a pixel of the correlation image that corresponds to the pixel of the strain image;
identifying a pixel value for the identified pixel of the correlation image, wherein the pixel value is a correlation value in a predetermined range;
comparing the identified pixel value with a predetermined threshold;
rendering the pixel of the strain image completely transparent in response to the identified pixel value not satisfying the predetermined threshold, creating the modified strain image pixel;
rendering the pixel of the strain image one of semi-transparent or opaque in response to the identified pixel value satisfying the predetermined threshold, creating the modified strain image pixel; and
superimposing the modified strain image pixel over the corresponding B-mode image pixel.

11. The method of claim 10, further comprising:
receiving a signal indicative of a user change in the predetermined threshold, creating an updated threshold;
comparing the identified pixel value with the updated threshold;
rendering the pixel of the strain image completely transparent in response to the identified pixel value not satisfying the updated threshold, creating a modified strain image pixel;
rendering the pixel of the strain image semi-transparent or opaque in response to the identified pixel value satisfying the updated threshold, creating the modified strain image pixel; and
superimposing the modified strain image pixel over the corresponding B-mode image pixel.

12. The method of claim 1, further comprising:
identifying a pixel of the B-mode image;
identifying a pixel value for the identified pixel of the B-mode image;
comparing the identified pixel value with a predetermined threshold;
rendering a pixel of the strain image corresponding to the pixel of the B-mode image completely transparent in response to the identified pixel value not satisfying the predetermined threshold, creating the modified strain image pixel;
rendering the pixel of the strain image corresponding to the pixel of the B-mode image one or semi-transparent or opaque in response to the identified pixel value satisfying the predetermined threshold, creating the modified strain image pixel; and
superimposing the modified strain image pixel over the corresponding B-mode image pixel.

13. The method of claim 1, further comprising:
transmitting an ultrasound signal into a field of view in which a subject or object is disposed in;
receiving a set of echoes;
generating the B-mode based on the set of echoes;
applying compression to the subject or object;
receiving a set of compression echoes; and
generating the strain image and the correlation image based on the set of compression echoes.

14. A system, comprising:
a memory that stores elastography visualization algorithms;
a processor that executes at least one of the elastography visualization algorithms, based on a visualization mode of interest, causing the processor to at least one of render a pixel of a strain image transparent or not at all, wherein the strain image is displayed overlaid over a B-mode image.

15. The system of claim 14, wherein the elastography visualization algorithms include 2D mappings, which include strain image information and reliability image information.

16. The system of, the processor further:
renders the pixel based on at least one of a corresponding correlation image or a B-mode image.

17. The system of claim 16, the processor further:
sets a transparency of the pixel as a function of a correlation value of a corresponding pixel in the correlation image.

18. The system of claim 16, the processor further:
threshold the pixel value based on a predetermined threshold, wherein the processor renders the pixel completely transparent in response to a correlation value of a corresponding pixel in the correlation image being below the predetermined threshold.

19. The system of claim 16, the processor further:
threshold the pixel value based on a predetermined threshold, wherein the processor at least one of renders the pixel completely transparent or does not render the pixel in response to a pixel value of a corresponding pixel in the B-mode image being below the predetermined threshold.

20. An ultrasound imaging system, comprising:
a transducer array of transducer elements;
transmit circuitry that generates a pulse that excites at least a sub-set of the transducer elements to transmit an ultrasound signal in a field of view;
receive circuitry that receives echoes, which are generated in response to the ultrasound signal interacting with structure in the field of view;
an echo processor that processes the echoes, generating a B-mode image;
an elastography processor that processes the echoes, generating a strain image and a corresponding correlation image; and
a rendering engine that renders the strain image over the B-mode image, wherein the rendering engine renders the strain image based on at least one of a soft blend algorithm, a hard blend algorithm, or a B-mode priority algorithm,
wherein the soft blend algorithm causes the rendering engine to render a pixel of the strain image using a transparency level,
wherein the hard blend algorithm causes the rendering engine to render the pixel of the strain image completely transparent, and
wherein the B-mode priority algorithm causes the rendering engine to ignore the pixel of the strain image.
